(19) United States
(12) Patent Application Publication
(10) Pub. No.: US 2022/0037005 A1
(43) Pub. Date: Feb. 3, 2022

(54) SYSTEM AND METHOD FOR IMPLEMENTING REMOTE MEDICINE

(71) Applicant: Healthcare Integrated Technologies Inc., Knoxville, TN (US)

(72) Inventors: Kenneth M. GREENWOOD, Davenport, FL (US); Scott Michael BORUFF, Knoxville, TN (US); Jurgen VOLLRATH, Sherwood, OR (US)

(21) Appl. No.: 17/392,065

(22) Filed: Aug. 2, 2021

Related U.S. Application Data
(60) Provisional application No. 63/103,411, filed on Aug. 3, 2020.

(51) Int. Cl. G16H 40/20; G16H 10/60; G16H 20/40; G16H 40/40

(52) U.S. Cl. CPC G16H 40/20; G16H 40/40; G16H 20/40; G16H 10/60

(57) ABSTRACT

A method and system for implementing remote medicine, comprises providing a medical service allocation system that allows service providers to register as designated providers, and allow users to make service requests for any items or services associated with an illness, injury or surgery.

### FIELD OF THE INVENTION

The invention relates to remote medicine. In particular, it relates to a system and method for implementing a remote medicine infrastructure.

### BACKGROUND OF THE INVENTION

Remote medicine (Telemedicine) has become a common new phrase that is being heralded as the way medicine will be performed in the future. New approaches are being sought to diagnose and treat patients more efficiently, while reducing costs and exposure to pathogens by patients and medical staff.

However, to date, telemedicine is severely limited, often comprising a simple video call with the patient in order to make a rudimentary initial diagnosis prior to requiring a face-to-face office visit or a referral to a specialist.

### SUMMARY OF THE INVENTION

The challenge in implementing telemedicine has been in the creation of an effective, user-friendly system.

According to the present invention, there is provided a system, that allows the physician to make an effective diagnosis based on all of the patient's physiological parameters, and then allows the physician to prepare a treatment plan that can be remotely implemented, and if necessary, support remote medical intervention, including surgery at a patient's location, followed by post-surgery care.

Thus, according to the invention there is provided A method of providing remote medicine, comprising storing medical provider information in a database or other memory, credentials, and availability information, receiving a request from a requestor, for delivery to a specified location, of one or more of: medical sensors, patient supplies, pre-surgical preparation, surgical equipment, physician consultation, and patient care, identifying suitable medical providers to respond to the service request based on type of service, availability of the provider, and location of the provider, and allocating the service request to a provider.

The method may include storing address information for each provider and allocating a geographic region to the provider, and may further include identifying the real-time location of a provider.

The method may include notifying suitable medical providers in order to allow them to accept a request prior to allocating the service request.

The notifying of medical providers, or the allocating of the service request may include providing the details of the location for the service delivery, including a GPS designation superimposed on a map.

The method may include canceling the allocation of a service request and assigning it to another medical provider if the service request is not handled within a defined period of time.

The requestor may select a provider from the list of suitable providers, thereby defining the allocation of the provider.

The service request may be a request for one or more medical sensors specified in the request, and the request may be visually presented to the provider.

The medical sensors may be undefined, but may be subsequently defined by the medical provider based on one or more of: conversations with the patient, conversations with health-care providers at a home or facility of the patient, conversations with relatives or authorized persons of the patient, and conversations with a physician.

The conversations with a physician may include a video call with the physician, and involve conversations between the physician and one or more of: the patient, medical provider, and health-care provider at a home or facility. For purposes of this application the term physician is used to cover any clinician dealing with the requestor's health, whether it be the patient's primary care provider, nurse practitioner, or physician's assistant.

Defining the set of sensors required may includes diagnosing one or more conditions of the patient requestor based on the conversations with the physician and data captured by one or more of pre-existing sensors associated with the patient requestor, and sensors provided by the medical provider.

Further, according to the invention, there is provided a system for addressing service calls for the delivery of remote medical services, comprising: a server, a database for storing information about medical providers wishing to respond to service calls, a platform that defines a provider dashboard for capturing medical provider information, and a requestor dashboard for capturing service requestor information and for allowing the requestor to submit a request for services, a notifying communications system for notifying suitable medical providers of service requests, and a memory with machine readable logic defining an algorithm for determining suitable providers for a service call based on the location of the service, availability of providers, and location of providers.

The database may include information about each medical provider in the system regarding the type of medical service provided by the provider, including one or more of: medical sensors, patient supplies, pre-surgical preparation, surgical equipment, physician consultation, and patient care.

The provider dashboard may include least one of: at least one data capture pages, and an API interface for capturing data about the provider.

The requestor dashboard may include a map with the real-time location of providers or their representatives, in order to provide the requestor with an overview and details of each provider within a defined proximity to the requestor, as well as an estimated time of arrival for each provider if they were selected.

The dashboard may include the ability to select a provider, and the system may further include a requestor-provider communications system for communication between requestor and provider prior to, during and after selection by text, email, voice or other means once a provider is selected.

The notifying communications system may includes a software app providing the medical provider with details about the location of the requestor or other location to which the provider's services should be directed.

The requestor dashboard may be linked to the notifying communications system and may include real-time requestor location information to help the medical provider locate the requestor.

At least one of the notifying communications system, and requestor-provider communications system may include video call functionality, to facilitate the establishment of a video call with a patient's physician so as to assist the physician in diagnosing the condition of a patient, and define the required sensors required by a patient for future remote monitoring, diagnosis or treatment.

The system may further include a communications link for linking to a patient's electronic medical records (EMR), to extract data from the EMR.

The machine readable logic in the memory may define an algorithm for capturing a record of the communications with the patient's physician through one or more of: a video file, sound file, and transcript of the communication. This allows a record to be kept of the conversation, which can be uploaded into the patient's EMR.

### BRIEF DESCRIPTION OF THE DRAWINGS

FIG. 1 a schematic representation of one embodiment of a system of the invention,

FIG. 2 is a representation of one embodiment of a data capture page for capturing information on medical providers,

FIG. 3 shows one embodiment of a user interface for a User App according to the invention, and

FIG. 4 is a block diagram of one embodiment of a Local Receiver Device of the invention.

### DETAILED DESCRIPTION OF THE INVENTION

The present invention provides a method and system for supporting the end-to-end needs of a patient and coordinating the activities of all participants in a healthcare system. As mentioned above, a mere video interaction between physician and patient seldom allows for a final diagnosis to be made. A patient may define the symptoms he or she is dealing with, and the video image of the patient may give the physician an idea whether the situation is critical and requires a follow-up visit or a referral to a specialist. However, in most cases additional information is needed to diagnose a condition.

Numerous physiological sensors and monitors have been developed that can be applied or used by a patient for purposes of measuring a wide variety of physiological parameters, such as blood pressure, electrocardiogram data, blood-oxygen levels, insulin levels, etc. However, these sensors and monitors invariably operate on proprietary platforms, leaving the patient with a bewildering panoply of options, some of which may be entirely superfluous or inadequate for the needs of the physician in diagnosing a particular ailment.

Insofar as further treatment is required following a diagnosis, the fallback position has been to either admit the patient to a hospital or provide the patient with a script of drugs and materials (e.g., bandages, splints, boots, crutches, walkers etc.) that the patient then has to collect at a pharmacy or medical supplier.

Surgical intervention invariably requires admission to a clinic or hospital, which exposes the patient to pathogens in the hospital, and furthermore, constitutes the most significant cost item in a surgery, and which may only be partially covered by a patient's insurance.

Post-surgical recuperation may add to the costs by requiring additional time in a hospital or several days or weeks at a care facility before the person is released to return home.

The present invention proposes a different approach that avoids the frequent need for a physician and support staff to attend to a patient at the physician's offices or at a clinic or hospital.

One aspect of the present invention, therefore, includes the coordination of medical service providers (also referred to herein simply as medical providers) who specialize in various aspects involved in the diagnosis and treatment of a patient, in order to provide home healthcare on an as-needed basis.

These medical service providers, include companies supplying medical sensors and monitoring equipment, patient care providers, medical support personnel involved in pre- and post-surgery activities, and physicians and medical personnel involved in the diagnosis and actual treatment of a patient, either with or without the need for surgery.

One embodiment of a such a system comprises a platform for making a service request and allocating services to medical providers according to spatial and temporal availability and according to type of service. The system includes means for communicating the allocation to the medical provider, e.g., by phone or text or by including a native application (App) that the provider downloads. In the embodiment where a provider App is included, the App may define a dashboard for the provider to enter his or her details, as well as a communications means for notifying the provider of a service request.

In one embodiment the dashboard includes a requestor dashboard, a physician dashboard and a provider dashboard to provide the parties with a visual overview of their respective commitments and service tasks that are to be met, estimated timelines and, in the case of the provider, a completion confirmation. This serves not only to define objectives that need to be met according to a timeline to ensure accountability, it allows activities to be coordinated with the selected provider or amongst multiple providers where multiple successive services need to be scheduled, e.g. pre-surgery preparation of a home, delivery of the necessary equipment, performance of the surgery, and post-surgery care and rehabilitation of the patient.

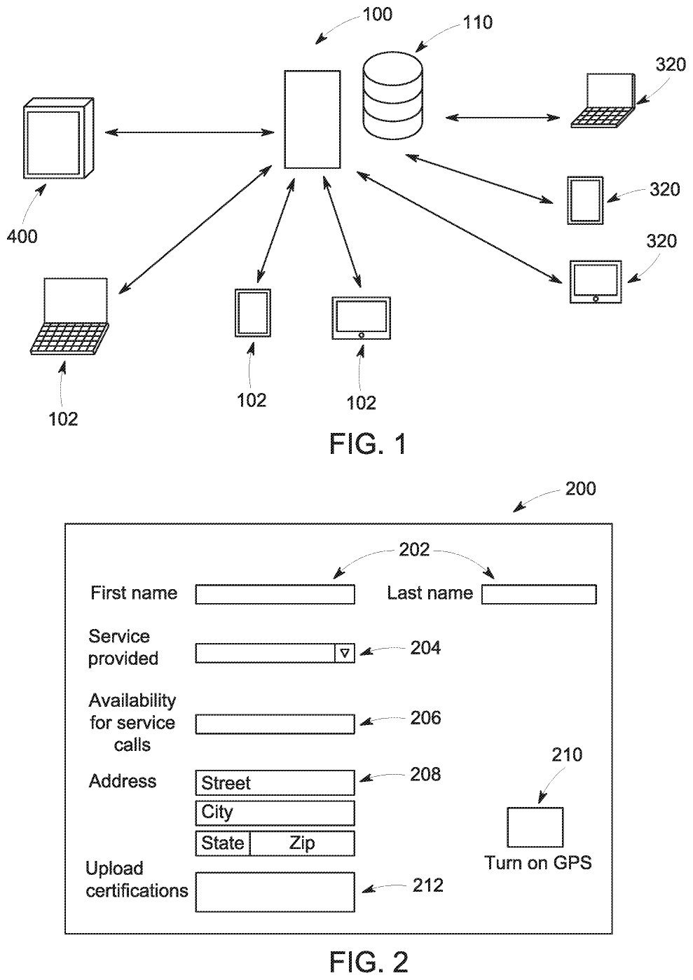

FIG. 1, shows an embodiment of one such system. Medical providers access a server 100, using their laptops, tablets, smart-phones, or other computing device 102 and interact with a web site via a browser, or access similar functionality as defined by a native software application (App) that is downloaded. The server 100, in one embodiment is implemented as a cloud server but may also be implemented as a centralized server configuration.

In one embodiment, medical providers initially sign up for inclusion into the system, via a portal, wherein the system in this embodiment includes the server 100 that defines a portal that provides a sign-up and log-in page. The medical providers are then presented with a profile capture page allowing them to enter their details.

One embodiment of a profile capture page 200 is shown in FIG. 2, which includes:
Name fields 202,
A drop-down menu 204 for selecting one or more services that they provide, which is discussed in greater detail below,
A temporal availability field 206 that allows providers to define the days and times that they are available in a particular week or month. In the present embodiment the temporal availability field 206 opens up a calendar when clicked, to allow a medical provider to select days and times, either on a day-by-day basis or for an entire week, month or year.
An address selection field 208 to define a geographic region assigned to the provider.
A GPS tracking button 210 that synchronizes the provider's cell phone GPS with a system tracking engine. This allows the system to capture and track the location of each medical provider's location for real-time location information in order to identify the provider's spatial availability.
A document upload field 212 for uploading Board certifications, Degrees, Diplomas, Certificates and Certifications, and other documents confirming the provider's credentials, or specifying a member number designating the provider as a member in good standing of a medical authorizing body.

As mentioned above, in the present embodiment the medical providers provide details of the type of service they provide and are allocated to one or more groups, for example, but not limited to:
1. Medical Sensor/Device Suppliers (also referred to herein as patient monitoring equipment),
The medical sensors/monitors/devices may include monitors for monitoring the movement and activities of a patient, sensors for measuring specific physiological parameters such as ECG, blood pressure, blood-oxygen levels, breathing monitors, blood-glucose level sensors, etc, and medical devices for taking samples of bodily fluids (blood, urine, saliva) or for administering medication, e.g., insulin delivery devices.
2. Providers of Patient Supplies (including drugs and wound care materials, and equipment),
The patient supplies may, for instance, include medication, wound care, and equipment, for post-operation, post-injury, or illness treatment. Equipment may include temporary or permanent items, e.g., crutches, braces, boots, wheelchairs, walkers, etc.
3. Pre-surgical Prep (for providing surgical preparation of a home),
Pre-surgical preparation thus includes the service of cleaning a home or continuous care facility and providing it with surgery support materials: tables, surgical bed, trays, lighting, equipment, e.g., scalpels, clamps, cauterizers, etc., and materials, e.g., sutures, swabs, gauze, bandages, disinfectants, etc.
4. Surgical Equipment Providers (for delivering and setting up surgical equipment).
Surgical equipment may include some or all of the material and equipment defined as surgical support materials under Pre-surgical Prep, as well as robotic surgery equipment, such as the Da Vinci robot with its cameras and monitors to allow some or all of the surgical procedures to be performed remotely.
5. Patient Care Support (comprising post injury or illness rehabilitation, including wound care and psychological support),
Patient Care Support thus includes services for providing physiological care, e.g., wound care or physiotherapy for rehabilitating a patient, after an injury or surgery, psychological support, and other forms of home care and support, including permanent care services for people that are incapacitated by injury or age.
6. Physician
The term physician is used herein for purposes of convenience to include all medical practitioners qualified to perform a diagnostic or treatment function, including military medical doctors, nurses, paramedics etc.

The system includes a memory, which in this embodiment is implemented as a database 110 (FIG. 1) for storing the medical provider information discussed above: name, service provided, temporal availability information (a schedule for when the provider is available), address, GPS tracking (to define spatial availability) and credentials. In one embodiment the interface accessed by a provider for entering the provider information is implemented as a portal accessible over the Internet, allowing the provider to enter and update relevant information.

In one embodiment, the system includes a software application (App) for medical providers, which allows them to enter their information, and also serves as a platform for notifying them whenever there is a service call. For ease of reference and to distinguish this App from the App discussed below for users requesting a service, the medical provider App will also be referred to herein as a Service Connectivity App or provider App.

As is discussed in greater detail below, a typical service request may be a request made by a physician or a patient or care-provider, requesting that a particular service be provided to a defined patient at a specified location. The process thus involves the server 100 receiving a service request for delivery of materials or services to a specified location, the materials or services including one or more of: medical sensors, patient supplies, pre-surgical preparation, surgical equipment, and patient care. One or more suitable medical providers are identified from the database 110, based on the type of service requested, the availability of the provider, and location of the provider. The service request is then allocated to one of these medical providers. In one embodiment the allocation is also dependent on the number of previous service requests that have been allocated to the available service providers over a certain time frame, in order to ensure a fair distribution of service calls.

In one embodiment, in order to make a service request, a user, e.g. a patient or physician, can accesses a user dashboard or physician dashboard, respectively, by downloading an App to their smart phone, or they can access a website via a browser in order to request a service. For ease of discussion, one embodiment of a patient user App will be discussed below.

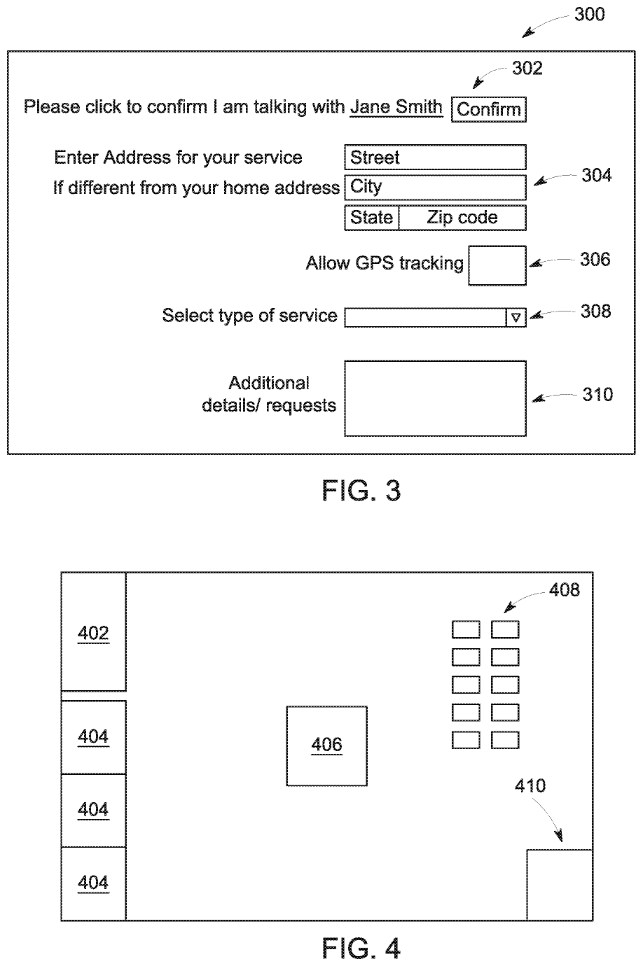

FIG. 3 shows a user interface (UI) for one embodiment of a User App 300. The User App 300, when downloaded may include a sign-on step requesting user details (name, address, and credit card information for on-line payment of services), and thereafter establishes user communication between the user's device 320 (smart phone, laptop, tablet, etc) and the server 100 as shown in FIG. 1.

Once signed on a user can make service requests through the User App. The User App confirms the identity of the user via a user confirmation field 302. The system defaults to the name of the user that registered for the app and requires the user to click to confirm: “Please Click to confirm that I am talking with . . . ”.

The user is prompted to verify the service address, which defaults to the user's home address and requires the service address to be entered if different from the user's home address (field 304).

For ease of locating the user's location, the user is encouraged to allow GPS tracking using the user's smart phone GPS (field 306)

Field 308 asks the user to specify the type of service by selecting from a drop-down menu. (“If you know the type of service you need, please select it here:”)

A generic description field 310 asks the user to describe their needs or concerns. Once the order is confirmed by clicking the CONFIRM button 312, the service request is allocated to a suitable medical provider, as discussed above.

The time flexibility of the present system allows not only permanent medical providers to respond to service requests, but also allows medical providers wishing to do freelance work, to sign up as a medical provider, download a Service Connectivity App, and be available during the time slots specified by them.

In a typical scenario, a freelancing Medical Provider signs up to the system to become a medical provider under the system.

The provider is led through the registration process as discussed above. Once they have provided their details and downloaded the Service Connectivity App they are ready to receive service calls for delivering one or more specified services.

A request may be initiated by a requestor, who may be a patient, a patient's general care provider, CCRC (Continuous Care Retirement Community), nursing facility, pharmacy, discharge nurse at a hospital, patient's family member, patient's physician, etc.

The system checks a database of registered medical providers to generate a list of one or more suitable providers to handle the call based on the type of service requested, availability of the providers, and proximity of the providers to the requesting location. In one embodiment, the system notifies the suitable providers via the Service Connectivity App and allows them to accept a service request. Once a provider accepts a call, they become the designated medical service provider for that service request and are required to provide the service within a pre-defined time period, to avoid being canceled for the service and opening up the service request to other available providers. If a provider is canceled, the system may employ an optimization algorithm, for example based on a rating, cost, value, or other subjective or non-subjective measurement to assign another provider or may notify the requestor of the cancelation with the option to make a new selection.

In another embodiment the requestor makes the initial choice based on the list of suitable providers generated by the system. The selected provider is then notified and becomes the designated provider for that service, once he or she accepts the request.

For example, a provider of physiology-monitoring sensors (medical sensors) could be notified of a service request by a patient located at an address located in the vicinity to where the provider is located and corresponding in the region that has been allocated to the provider. The service in this case would include delivering the sensors/monitors/devices to the patient, and leading the patient or care-giver through the steps of using the sensors that are delivered. In the case of some sensors it may require installing the sensors in the patient's home or suite and integrating the data communication with the server 100.

In one embodiment, the types of sensors/devices needed by a patient will be identified in the Service Connectivity App as part of the service allocation.

In another embodiment the medical service provider will have to make a determination about the types of sensors required. This may include discussions with the patient, caregivers, or family members of the patient. It may also require establishing a video call with the patient's primary care physician or other designated physician. In a preferred embodiment, both the Service Connectivity App and the User App include video call functionality to allow a video call to be established with a physician or other designated person directly from either one of the software applications.

Insofar as a patient or a patient's residence already includes one or more sensors for use in monitoring the patient or for diagnosing the patient's condition, data from the sensors, which is being fed to the server 100, may be made available to the physician (e.g., via the physician's Service Connectivity App) to supplement the video call discussion, in order to inform the physician as to the best set of additional sensors and devices needed by the patient for ongoing or future monitoring, diagnosis or treatment. The medical provider may also hook up additional sensors brought along in response to the service request, by establishing a communication link between the sensors and the server 100 and, where necessary, applying or attaching the sensors to the patient. This allows the physician to make a better diagnosis and help identify the optimum set of sensors that should form part of the service request.

One aspect of the present invention includes consolidating the operability of medical sensors from different providers for integration with the system of the invention, in order to capture and analyze data from multiple medical sensors and monitors. This provides a physician with a comprehensive physiological picture of a patient based on multiple data sources for better diagnosis. Many third-party medical sensors currently make use of Bluetooth connections to a smart phone, which then serves as communication device to submit data to a cloud server. Other medical sensors may include an integrated Wifi or cell phone communications system.

In one embodiment Bluetooth data, or other radio data from the sensor can be captured by a Local Receiver Device depicted by reference numeral 400 (also shown in FIG. 1), which includes a Bluetooth receiver 402 and multiple other radio receivers 404 to support the various other radios used by any of the sensors that are being supported, a processor 406, a memory 408 for buffering data received from one or more medical sensors, and a Wifi transceiver 410 for communicating with the server 100. Data from each medical sensor will be identified by header information in the message received from each sensor, which will be unique for each sensor. The Device 400 will thus be able to communicate each sensor's data to the server 100 via an Internet connection.

Another aspect of the present invention involves training and certifying medical service providers of medical sensors to allow them to install and implement the various sensors approved for use by the system of the present invention.

In one embodiment one or more sensors may be pre-packaged into a Condition-Specific set of sensors for monitoring a pre-defined condition, e.g. heart congestion, sleep apnea, blood-glucose levels, etc.

In this embodiment, the medical providers may each be provided with several pre-packaged Condition-Specific sets of sensors/monitors/devices (also referred to herein as a MedSet) to cover various conditions that may need to be addressed during a service call.

In the service call embodiment discussed above, involving a medical sensor provider, the Service Connectivity App will instruct the designated service provider with details of the location, typically coupled with GPS tracker information of the location of the service call. As discussed above, it may also specify the MedSet to be delivered and the amount of the remuneration to the medical provider. As discussed above, where the service call does not pre-define the MedSet, the provider may be required to ascertain from the patient, care-provider, family member, or other person that has observed the patient, to identify the likely MedSet required by the patient. As discussed above, the choice of MedSet may require a conversation with a physician, e.g., the patient's primary care physician in a video call while the provider is with the patient, to allow the physician to ask health-related questions and make a determination on what sensors would be appropriate for continued monitoring the patient's health condition.

The provider then leads the patient or caregiver through the steps of using the various sensor/devices that will be left with the patient or care-giver, and completes his or her service call.

The patient or care-facility may be billed at the time of the service call, e.g., by means of a credit card on file (collected when the patient downloaded the User App), or may be billed subsequently for the service call and the cost of the MedSet.

The service delivered by the provider, thus comprises delivery of a MedSet and teaching of a patient or caregiver how to use the various sensors/devices in the MedSet. It may also require the provider to make a determination of which MedSet to deliver based on conversations with the patient and/or associated people, either with or without the assistance of a physician. In one embodiment the provider receives a flat delivery and teaching fee depending on the nature and complexity of the MedSet (including the number and types of sensors, monitors, and devices in the MedSet), and a MedSet determination fee insofar as the type of MedSet is not predefined at the time of the service request.

It will be appreciated that the embodiments discussed above comprise only some examples of how to implement the system of the present invention and the interaction with users and medical service providers. The system may therefor include other ways of implementing the system, without departing from the scope of the invention.

What is claimed is:

1. A method of providing remote medicine, comprising
storing medical provider information in a database or other memory, credentials, and availability information,
receiving a request from a requestor, for delivery to a specified location, of one or more of: medical sensors, patient supplies, pre-surgical preparation, surgical equipment, physician consultation, and patient care,
identifying suitable medical providers to respond to the service request based on type of service, availability of the provider, and location of the provider,
and
allocating the service request to a provider.

2. The method of claim 1, further comprising storing address information for each provider and allocating a geographic region to the provider.

3. The method of claim 2, further comprising identifying the real-time location of a provider.

4. The method of claim 2, wherein identified suitable medical providers are notified in order to allow them to accept a request prior to allocating the service request.

5. The method of claim 4, wherein the notifying of medical providers, or the allocating of the service request may include providing the details of the location for the service delivery, including a GPS designation superimposed on a map.

6. The method of claim 2, further comprising canceling the allocation of a service request and assigning it to another medical provider if the service request is not handled within a defined period of time.

7. The method of claim 2, wherein the requestor selects a provider from the list of suitable providers, thereby defining the allocation of the provider.

8. The method of claim 1, wherein the service request is a request for one or more medical sensors specified in the request, and the request is visually presented to the provider.

9. The method of claim 1, wherein the service request is a request for an undefined set of medical sensors, which is subsequently defined by the medical provider based on one or more of: conversations with the patient, conversations with health-care providers at a home or facility of the patient, conversations with relatives or authorized persons of the patient, and conversations with a physician.

10. The method of claim 9, wherein the conversations with a physician include a video call with the physician, and involve conversations between the physician and one or more of: the patient, medical provider, and health-care provider at a home or facility.

11. The method of claim 10, wherein defining the set of sensors required includes diagnosing one or more conditions of the patient based on the conversations with the physician and data captured by one or more of pre-existing sensors associated with the patient, and sensors provided by the medical provider.

12. A system for addressing service calls for the delivery of remote medical services, comprising:
a server,
a database for storing information about medical providers wishing to respond to service calls,
a platform that defines a provider dashboard for capturing medical provider information, and a requestor dashboard for capturing service requestor information and allow the requestor to submit a request for services,
a notifying communications system for notifying suitable medical providers of service requests, and
a memory with machine readable logic defining an algorithm for determining suitable providers for a service call based on the location of the service, availability of providers, and location of providers.

13. The system of claim 12, wherein the database includes information about each medical provider in the system regarding the type of medical service provided by the provider, including one or more of: medical sensors, patient supplies, pre-surgical preparation, surgical equipment, physician consultation, and patient care.

14. The system of claim 13, wherein the provider dashboard includes least one of: at least one data capture pages, and an API interface for capturing data about the provider.

15. The system of claim 12, wherein the requestor dashboard includes a map with the real-time location of providers or their representatives, in order to provide the requestor with an overview and details of each provider within a defined proximity to the requestor, as well as an estimated time of arrival for each provider if they were selected.

16. The system 15, wherein the requestor dashboard includes the ability to select a provider, and the system further includes a requestor-provider communications system for communication between requestor and provider prior to, during and after selection by text, email, voice or other means once a provider is selected.

17. The system of claim 16, wherein the notifying communications system includes a software app providing the medical provider with details about the location of the requestor or other location to which the provider's services should be directed.

18. The system of claim 17, wherein the requestor dashboard is linked to the notifying communications system and includes real-time requestor location information to help the medical provider locate the requestor.

19. The system of claim 16, wherein at least one of the notifying communications system, and requestor-provider communications system includes video call functionality, to facilitate the establishment of a video call with a patient's physician, so as to assist the clinician in diagnosing the condition of a patient, and define the required sensors required by a patient for future remote monitoring, diagnosis or treatment.

20. The system of claim 19, further including a communications link for linking to a patient's electronic medical records (EMR), to extract data from the EMR.

21. The system of claim 20, wherein the machine readable logic in the memory defines an algorithm for capturing a record of the communications with the patient's physician through one or more of: a video file, sound file, and transcript of the communication.
